(19) United States
(12) Patent Application Publication
(10) Pub. No.: US 2011/0208103 A1
(43) Pub. Date: Aug. 25, 2011

(54) Disposable medical article with multiple adhesives for skin attachment

(76) Inventor: Boris Leschinsky, Waldwick, NJ (US)

(21) Appl. No.: 11/265,656

(22) Filed: Nov. 2, 2005

Related U.S. Application Data
(63) Continuation of application No. 10/619,966, filed on Jul. 15, 2003, now abandoned.

(51) Int. Cl. A61L 15/58; C09J 7/02; A61L 15/16

(52) U.S. Cl. 602/57; 428/41.7

(57) ABSTRACT

A disposable medical article for skin attachment is provided with two or more stacked layers of pressure-sensitive adhesives with progressively higher skin peel strength separated by protective covers therebetween to accommodate a wide range of skin conditions such as wet or oily skin while reducing the risk of skin irritation. A lower strength adhesive is preferably exposed first by removing its protective cover to attach the article to the skin. If the article separates from the skin, the next protective cover is removed revealing an additional more aggressive adhesive layer so the article may be reattached. Alternatively, the more aggressive adhesive can be exposed right away by removing another adhesive layer and its protective cover as a unit. The article may be used advantageously for wound dressings, EKG electrodes, hemostasis patches, ostomy bag attachments and alike to provide a more secure attachment over a wide range of patients.

### BACKGROUND OF THE INVENTION

The present invention relates generally to pressure-sensitive adhesive medical articles for skin or nail attachment such as a wound dressings, EKG electrodes, catheter securement devices and alike. More particularly, the article of the invention utilizes more than one layer of adhesive to optionally vary the attachment force to the skin if required.

A number of medical articles and devices are applied to the skin or nail of a patient using a pressure-sensitive adhesive on one side thereof. For the purposes of this description, these devices are collectively called “an article” and include but not limited to the following examples: wound covers and protective dressings; various skin adhesive strips, tapes, including foam tapes; skin electrodes including EKG electrodes; catheter, tube, and cannula skin attachment and securement devices; hemostasis patches and alike; transdermal medication delivery patches; transcutaneous ports including those equipped with a skin attachment skirt; active compression/decompression CPR devices utilizing a skin attachment patch for pushing and pulling on the chest. In addition, other medical devices are included in the scope of the present invention, which contain a skin attachment component for securing the device to the patient. Such devices include disposable absorbent diapers, sanitary napkins, pantiliners, tampons, perspiration pads, adult incontinence devices and the like to be attached directly to the skin of the patient.

The adhesive layer in a typical article of the prior art is covered with a releasable protective liner, which is removed prior to the application of the article to the skin of a patient. The adhesive layer itself can cover the entire surface of the article or only a portion thereof depending on its function. For example, the central portion of the article may contain an absorbent pad or an active ingredient designed to effect the underlying skin. In that case, the adhesive is typically applied only on the periphery of the article.

It is a functional requirement that the article adheres well to the skin of a patient across a wide range of skin conditions and in various regions of the body. Frequently, the skin of a patient can be dry or oily, contain wrinkles, contain wet areas, covered with hair, etc. On the other hand, many applications call for the adhesion to the sensitive areas of the body such as in pediatric applications, etc. Therefore the adhesive of the article has to be more aggressive to accommodate the most difficult skin conditions on the one hand and at the same time has to be less aggressive to prevent skin irritation and injury on the other hand as well as facilitate a painless removal.

The adhesives used to secure the article in place on a patient are typically chosen to be on a fairly aggressive side to prevent an unwanted release from the skin. That tends to cause skin irritation for some patients. At the same time, for some other patients the same article may occasionally or even frequently fall off the skin anyway requiring a replacement, or multiple replacements.

The prior art in the general field of adhesives for attachment to the skin is particularly developed in the field of articles such as band-aids, plasters and bandages. Such wound covering absorbent articles are mostly adhered to the skin where prior to application of the absorbent article bodily hair can be removed or where little hair grows.

In order to provide the desired level of adhesion of such bandages, the prior art typically discloses the use of certain adhesives having very high cohesive strengths such as rubber-based adhesives and acrylics. These adhesives are then applied as thick layers to maximize the adhesive force by which the bandage is secured to the skin of the wearer.

U.S. Pat. No. 4,699,146 discloses hydrophilic elastomeric pressure-sensitive adhesives suitable for use with ostomy devices, bandages, ulcer pads, sanitary napkins, diapers, and athletic padding. The adhesive comprises at least 1 radiation cross-linked organic polymer and an adhesive plasticizer.

GB Patent No. 2,115,431 discloses adhesives for bandages, wounds or burn dressings, EKG adhesives, sanitary napkins, diapers and ulcer pads. The adhesive comprises an irradiation cross-linked organic polymer such as polyvinylpyrrolidone and an adhesive plasticizer.

Adhesion to wet skin is addressed in WO 98/03208, which discloses medical pressure-sensitive adhesives, which can adhere to dry or wet skin and which comprise a mixture of hydrophilic (meth)acrylate copolymer containing tertiaryamino groups, a hydrophilic (meth)acrylate copolymer containing carboxyl groups, carboxylic acids and a cross-linking system.

U.S. Pat. No. 6,544,642 describes a disposable absorbent article with improved adhesives designed to ensure the skin attachment in a variety of difficult conditions such as for an oily or greasy skin as well as for wet or moist skin. The ratio of dry peel strength to greasy peel strength is claimed to be within the range of 1:1 to 1:0.2 and the adhesive contains a polymer forming a three-dimensional matrix as well as a hydrophilic component and a hydrophobic component. Viscosity and thickness are also defined.

Finally, U.S. Pat. No. 6,461,467 discloses a dressing with multiple adhesives, some containing a biologically active agent, these adhesives being exposed to the skin all at the same time. No flexibility is therefore provided in choosing the adhesive strength for a particular skin condition.

The need therefore exists for an article capable of accommodating a wide range of skin conditions but with reduced risk of causing a skin irritation. The need also exists to provide an article with a choice of adhesive strength depending on a skin condition.

### SUMMARY OF THE INVENTION

Accordingly, it is an object of the present invention to overcome these and other drawbacks of the prior art by providing a novel disposable medical article having more than one layer of adhesives with multiple bondage strength.

It is another object of the present invention to provide a medical article capable of providing varying levels of skin or nail attachment depending on the choice of the user.

It is a further object of the present invention to provide a medical article capable of secure attachment to the skin of a patient over a wide range of skin conditions and with reduced risk of causing skin irritation or damage.

It is yet a further object of the present invention to provide a medical article with improved painless removal.

The article of the invention has a backing with a bottom surface facing the patient. A layer of first adhesive is applied to that surface. A layer of a first protective cover is releasably placed over the first adhesive layer to protect thereof from exposure to the patient. A layer of a second adhesive is applied to the bottom of the first protective cover and in turn is covered on the other side with the second protective cover. Importantly, both protective covers may have protruding tabs so either one can be removed from the article depending on the choice of the user. Also, the attachment strength (or peel strength) of the first protective cover to the first adhesive layer is preferably stronger than that to the second adhesive layer, which in turn is preferably stronger than the attachment strength of the second protective cover to the second adhesive layer. Therefore, an increasing succession of attachment strengths is formed to ensure a reliable attachment of the article to the skin of the patient.

In use, either one of adhesive layer can be exposed at first to the patient. In a preferred method of use, a second, less aggressive layer of adhesive is exposed first by removing a second protective cover from the article. In case of failure, the first more aggressive layer can then be easily exposed and the article can be reattached to the skin. Optionally, for difficult skin conditions, the user may choose to expose first adhesive layer from the beginning and ensure a firm attachment property of the article of the invention.

### BRIEF DESCRIPTION OF THE DRAWINGS

A more complete appreciation of the subject matter of the present invention and the various advantages thereof can be realized by reference to the following detailed description in which reference is made to the accompanying drawings in which:

FIG. 1 is a side cross-sectional view of the article of the invention, and

FIG. 2 is an exploded view of the same article showing various layers of adhesives and protective covers thereof.

### DETAILED DESCRIPTION OF THE PREFERRED EMBODIMENT OF THE INVENTION

A detailed description of the present invention follows with reference to accompanying drawings in which like elements are indicated by like reference letters and numerals.

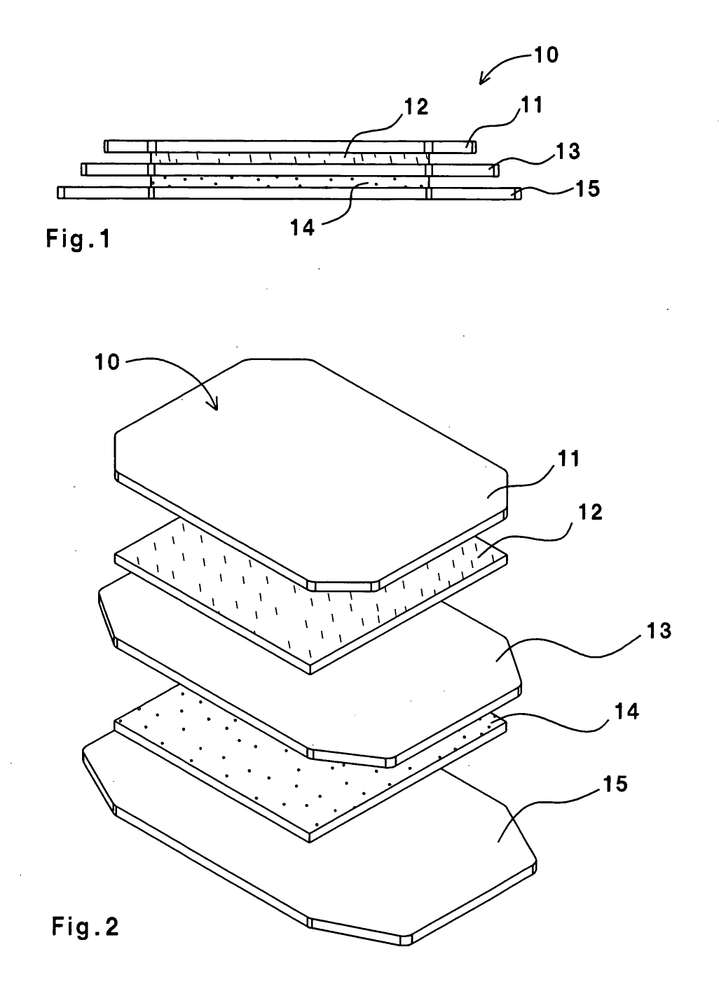

FIGS. 1 and 2 depict an illustrative embodiment of one medical article according to the present invention. The article 10 has a backing 11 having a top surface and a bottom surface. The bottom surface is defined as that surface that is designed to face the patient when the article 10 is applied and is typically the surface of the article on which any pressure-sensitive adhesives used to secure the article 10 are exposed. The backing 11 is preferably a polymeric film or sheet that is optionally contact transparent. Various design features may be optionally associated with the top surface of backing 11 but are not shown on the drawings. Examples of such features include attachment means to secure a catheter, a tube, or a medical device to the skin of the patient as well as other medical treatment elements.

The backing 11 is also preferably conformable to anatomical surfaces. As such, when the backing 11 is applied to an anatomical surface, it conforms to the surface even when the surface is moved. The preferred backing is also conformable to anatomical joints. When the joint is flexed and then returned to its initial position, the backing stretches to accommodate the flexion of the joint, but is resilient enough to continue to conform to the joint when the joint is returned to its initial condition. A description of this characteristic of backings preferred for use with the present invention can be found in U.S. Pat. Nos. 5,088,483 and 5,160,315.

A description of some backings that may be preferred for use in the medical dressings of the present invention can also be found in the U.S. Pat. Nos. 5,088,483 and 5,160,315, as well as European Patent Application Publication No. EP 0 437 944.

Particularly preferred backing material may be selected from the group of elastomeric polyurethane, copolyester, or polyether block amide films, or combinations thereof. These materials combine the desirable properties of resiliency, high moisture vapor permeability, and transparency that may be preferred in the design of the article backing. Also, although the backings are depicted below as monolayer articles, it will be understood that they could include multiple layers as described in, e.g., European Patent Application Publication No. EP 0 437 944.

Pressure-sensitive adhesives that can be used in the medical articles of the present invention include adhesives that are preferably compatible with human or animal skin or nails, more preferably those that are of the class known as “hypoallergenic” adhesives. Examples of some adhesives useful in connection with the invention include, but are not limited to the acrylate copolymers described in U.S. Pat. No. RE 24,906, particularly a 97:3 iso-octyl acrylate:acrylamide copolymer. Also useful is an 70:15:15 isooctyl acrylate; ethyleneoxide acrylate; acrylic acid terpolymer, as described in U.S. Pat. No. 4,737,410. Other useful adhesives are described in U.S. Pat. Nos. 3,389,827; 4,112,213; 4,310,509; and 4,323,557; as well as UK Patent No. 1280631 (see, e.g., polyvinyl ether adhesives) and European Patent Nos. 35399 and 51935. Some useful pressure-sensitive adhesives may include bioactive agents as described in, e.g., U.S. Pat. Nos. 4,310,509; 4,323,557; 5,614,310; and 5,908,693. Some preferred bioactive agents may be antimicrobial agents to enhance wound or catheter site infection control.

The layers of pressure-sensitive adhesives located on the backings of the medical article of the invention may optionally be chosen so as to transmit moisture vapor at a rate greater than or equal to that of human skin. While such a characteristic can be achieved through the selection of an appropriate adhesive, it is also contemplated in the present invention that other methods of achieving a high relative rate of moisture vapor transmission may be used, such as, e.g., pattern coating the adhesive.

In addition to moisture vapor permeability and hypoallergenic nature, it may also be preferred that the adhesives exhibit high initial tack upon application to the skin or the surface of a nail. One such pressure-sensitive adhesive is described in U.S. Pat. No. 5,849,325 incorporated herein by reference, and other useful adhesives may include polyvinyl ether adhesives as discussed in, e.g., UK Patent No. 1280631. One advantage of an adhesive exhibiting high initial tack is additional securing of, e.g., a catheter by the article may be more quickly enhanced as opposed to adhesives that have a lower initial tack.

When provided as a part of the medical article (before delivery), suitable protective covers are available from a variety of manufacturers in a wide variety of proprietary materials. Those skilled in the art will normally test those covers or liners in simulated use conditions against an adhesive of choice to arrive at a product with the desired release characteristics. The materials used to supply the protective covers for the flexible backing article manufactured according to the present invention are preferably substantially more rigid than the backing to ensure easy removal when required.

In its most general form, the article 10 has a backing 11 having a bottom surface. The first adhesive layer 12 is disposed onto the bottom surface of the backing 11 and in turn has a first lower surface facing the patient. The first adhesive layer 11 is covered with a first protective cover 13 attached to the first lower surface to prevent its exposure to the patient before use. The first lower surface of the first protective cover 13 facing the patient is exposed in turn to the second adhesive layer 14 disposed thereon. The other side of the second adhesive layer 14 facing the patient forms the second lower surface and is covered with the second protective cover 15 for the same purpose.

The shape of protective covers 13 and 15 may extend beyond the adhesive layer as shown on the drawings. The tabs protruding outside the boundaries of the article 10 may be used to remove the covers 13 and 15 from the article 10 just before use. It is preferred to provide the tabs of the second cover 15 to be longer than the tabs of the first cover 13 so as to facilitate easy access thereto. Alternatively, these tabs can face in different directions and may also be optionally color-coded to enhance intuitive understanding of their function. Also, more than one part of layer may constitute a single protective cover so as to further enhance its removal. A typical overlaying in the middle tabs such as those used for protective covers in Band-Aids is one example of such multilayer design contemplated within the scope of the invention.

Each layer of adhesive and each layer of protective cover are characterized by the attachment strength therebetween. Importantly, the materials of adhesives and protective covers are chosen such that the peel strength between the second cover 14 and the second adhesive layer 15 is the lowest to facilitate their easy separation.

The second adhesive layer material is preferably chosen to provide a skin peel strength to be less than a peel strength between it and the first protective cover 13 so as to ensure that the attachment strength of the article 10 to the skin is higher than that between the layers 14 and 13 thereof in case the second adhesive layer 14 is used for attachment to the patient.

The peel strength between the first protective cover 13 and the first adhesive layer 12 is preferably progressively higher than the skin peel strength of the second adhesive layer 14. At the same time, this strength is chosen lower that the peel strength between the first protective cover 13 and the second adhesive layer 14 to ensure easy removal from the patient of the article 10 in one piece. In fact, it is preferred that the first protective cover 13 is not separable from the second adhesive layer 14 at all.

The skin peel strength of the first adhesive layer 12 is preferably chosen to be higher than the skin peel strength of the second adhesive layer 14 to provide for more aggressive skin attachment when required. The skin peel strength is determined using standard test conditions known in the art such as a peel strength measured against a flat dry steel surface.

Finally, the attachment between the first adhesive layer 12 and the bottom surface of the backing 11 is the strongest of all forces so as to prevent any separation therebetween at any time.

In use, several options exist for the user of an article. In the most preferred method, the second protective cover 15 is removed first exposing a second adhesive layer 14 to the skin of a patient. Since the second adhesive is chosen to be of sufficient strength for most patients but not cause any skin irritation, the article is firmly pressed against the skin of the patient for attachment thereto. In a rare case of dislodgment of the article from the skin, the first protective cover 13 is then removed exposing the first more aggressive adhesive layer 12 and the article is reattached to the patient.

In an alternative method of use, especially for obviously difficult skin conditions, the first protective cover 13 may be removed right away along with the second adhesive layer 14 and the second protective cover 15 so that the first more aggressive adhesive layer is used for the attachment of the article 10 to the patient.

An important advantage of the article of the present invention is that since an additional adhesive layer is available, the main adhesive layer can be chosen to be less aggressive and therefore reduce the risks of skin irritation for some patients such as for children. The article therefore may be used for a wider patient population than other articles known in the prior art while at the same time providing for a more secure attachment.

Although the invention herein has been described with respect to particular embodiments, it is understood that these embodiments are merely illustrative of the principles and applications of the present invention. One useful design of the article of the invention is contemplated to include three or more layers of adhesives, each with its own protective cover to further enhance the choice of the user as to the level of peel strength desired for a particular application. Another useful application is for a non-medical area where the article of the invention may be used for attachment to surfaces other than skin where an ability to vary the peel strength may be desirable. It is therefore to be understood that numerous modifications may be made to the illustrative embodiments and that other arrangements may be devised without departing from the spirit and scope of the present invention as defined by the appended claims.

What is claimed is:

1. A medical article for skin attachment comprising:
a backing having a bottom surface,
a first adhesive layer disposed onto said bottom surface of said backing, said first adhesive layer having a first lower surface facing away from said bottom surface,
a first protective cover releasably attached to said first lower surface of said first adhesive layer, said first protective cover having a first cover lower surface facing away from said first lower surface,
a second adhesive layer disposed onto said first cover lower surface of said first protective cover, said second adhesive layer having a second lower surface facing away from said first cover lower surface, and
a second protective cover releasably attached to said second lower surface of said second adhesive layer.

2. The medical article as in claim 1, wherein said first adhesive layer comprises a first adhesive characterized by a first peel strength, said second adhesive layer comprises a second adhesive characterized by a second peel strength, said second peel strength being lower than said first peel strength.

3. The medical article as in claim 1, wherein said first protective cover comprising a material characterized by a peel strength between thereof and a first adhesive layer, said strength being higher than said second peel strength.

4. The medical article as in claim 1, wherein said first protective cover extends beyond said first adhesive layer to form at least one first tab to facilitate its removal.

5. The medical article as in claim 4, wherein said second protective cover extends beyond said second adhesive layer to form at least one second tab to facilitate its removal.

6. The medical article as in claim 5, wherein said second tab extends beyond said first tab.

7. The medical article as in claim 5, wherein said first and second tabs are color-coded.

8. A disposable article for attachment comprising:
a backing having a bottom surface,
a first adhesive layer disposed onto said bottom surface of said backing, said first adhesive layer having a first lower surface facing away from said bottom surface,
a first protective cover releasably attached to said first lower surface of said first adhesive layer, said first protective cover having a first cover lower surface facing away from said first lower surface,
a second adhesive layer disposed onto said first cover lower surface of said first protective cover, said second adhesive layer having a second lower surface facing away from said first cover lower surface, and
a second protective cover releasably attached to said second lower surface of said second adhesive layer.

9. The medical article as in claim 8, wherein said first adhesive layer comprises a first adhesive characterized by a first peel strength, said second adhesive layer comprises a second adhesive characterized by a second peel strength, said second peel strength being lower than said first peel strength.
